(19) United States
(12) Patent Application Publication
(10) Pub. No.: US 2001/0039010 A1
(43) Pub. Date: Nov. 8, 2001

(54) SAMPLE COLLECTION MEDIUM INCORPORATING MATERIAL FOR SAMPLE VISUALIZATION

(76) Inventor: LEIGH ALEXANDER BURGOYNE, MITCHAM (AU)

Correspondence Address: KENNETH I. KOHN, KOHN & ASSOCIATES, 30500 NORTHWESTERN HWY., SUITE 410, FARMINGTON HILLS, MI, 48334, US

(21) Appl. No.: 09/386,784

(22) Filed: Aug. 31, 1999

Related U.S. Application Data
(60) Provisional application No. 60/098,940, filed on Sep. 3, 1998.

(51) Int. Cl. C12Q 1/00; G01N 21/00; A61K 9/14; G01N 33/48; C12Q 1/68; G01N 31/22; G01N 15/06; G01N 33/00

(52) U.S. Cl. 435/6; 422/68.1; 422/56; 424/484; 435/4

(57) ABSTRACT

The present invention provides a dry solid medium with a visible tracer useful in the collection and tracking of biological materials in a form suitable for storage and subsequent analysis. In particular, the invention provides a dry solid medium with an inert visible material which, upon contact with a biological sample, indicates the area of the dry solid medium occupied by the sample. The invention also provides a dry solid medium including additional components which function in subsequent analysis of biological materials using, for example, PCR, reverse transcriptase initiated PCR, LCR, RFLP, or genetic hybridization. The invention further provides methods for using the dry solid medium of the invention.

### BACKGROUND OF THE INVENTION

1. Field of the Invention

The present invention is directed to a visualization system used in the collection of biological samples.

2. Description of Related Art

The collection, transport and storage of biological materials prior to analysis presents a number of problems to laboratory personnel. Multiple biological samples are routinely obtained for evaluation and analysis in a large variety of situations, a practice that necessitates the careful storage and tracking of these specimens. For example, samples containing genetic materials and other macromolecules to be analyzed are typically transported from the initial laboratory or place of removal to the place of analysis in a variety of forms including purified genetic material, liquid sample, frozen sample or dried onto paper. While all of these methods of transport have disadvantages, the transport of genetic material as a dried, purified genetic material is most desirable, but requires a high standard of technical assistance to be available at the place of removal from the human or animal. When technical assistance is not available at the place of removal, liquid samples such as saliva, urine or other unpurified samples are usually sent to a central facility where the genetic material and other macromolecules are subsequently purified and analyzed.

Transport of biological samples often involves the need for sterility of collection. The transport of liquid or frozen biological samples also demands temperature control and an appropriate transport system other than the regular postal system. This is true even before considering concerns about hygiene. In addition, problems with pathogens associated with biological samples generally rule out the transport of any potentially infectious liquid or frozen sample except under proper and expensive supervision.

Biological samples dried on filter paper is a favored alternative to the above procedures involving liquid or frozen samples due to recent advances in the procedures involving the extraction and isolation of macromolecules from dried sample spots in a form and in sufficient quantities for use in DNA analysis. Berlin, Y. A., et al., “Rapid Preparation of Genomic DNA from Dried Blood and Saliva Spots for Polymerase Chain Reaction,” Hum. Mutat. 1(3):260-261 (1992). While these protocols offer a number of advantages, they still suffer from a number of drawbacks. In particular, researchers are hindered by the inability to track substantially colorless samples which have been dried onto paper or a related medium.

There are a number of problems associated with difficulties in tracking slightly colored or clear liquid samples which have been dried onto paper or a related medium. In situations where samples may contain human pathogens, the inability to see where the infectious materials have been spotted on the medium creates an exposure hazard for laboratory personnel. In addition, in situations where low concentrations of biological materials require a comprehensive retrieval of the sample, the inability to visualize the boundaries of the biological sample decrease the chances of recovering an amount sufficient for analysis. Moreover, in any centralized facility which receives multiple samples for analysis of genetic materials, the logistical problems that come with the handling of relatively clear samples can be immense.

As the use of dry storage mediums for transporting biological materials increases, there is a concurrent increase in the need for a safe, convenient and minimally labor intensive means for tracking of substances contained in clear biological samples. Accordingly, it is desirable to have specific materials and methods for identifying and tracking these biological materials prior to analysis.

### SUMMARY OF THE INVENTION

The present invention provides a dry solid medium with a visible tracer useful in the collection, tracking and purification of biological materials in a form suitable for storage and subsequent analysis. The invention provides a dry solid medium for storing at least one biological sample, with the dry solid medium comprising a material that enables the visualization of the sample applied to the medium. In particular, the dry solid medium may comprise a pattern formed by an inert visible material where, upon application of a sample, the visible pattern is altered so that an area of the dry solid medium occupied by the sample is visible. For example, upon application of a liquid biological sample to surface of the medium, the fluid in the sample will solubilize the visible material which diffuses with the liquid sample so that an area of the dry solid medium occupied by the liquid sample is visible against the background of nondiffused material. In another aspect of the invention, the visible material is applied to the medium following deposition of a sample so as to reveal the position of the sample. The invention also provides a dry solid medium including additional components which function in subsequent analysis of biological materials using, for example, PCR, reverse transcriptase initiated PCR, LCR, RFLP, or genetic hybridization. As such, this invention provides a safe, convenient and minimally labor intensive apparatus and method for visualizing, tracking and analyzing biological macromolecules contained in biological samples.

One embodiment of the invention consists of a dry solid medium for storing at least one biological sample, this dry solid medium containing an inert visible material which, upon application of a liquid sample, will diffuse with the liquid so that an area of the dry solid medium occupied by the liquid is visible. In a more specific embodiment of the invention, the dry solid medium consists of a cellulose based paper. In preferred embodiment of the invention, the inert visible material consists of a pigment or dye such as colloidal carbon or metals, bromophenol blue or carminic acid. In a highly preferred embodiment, the pigment or dye forms a pattern on the dry storage medium such as a grid, a checkerboard or a series of repetitive dots.

The invention also provides a dry solid medium and visualization system having additional compositions included therein to facilitate the storage of a sample of genetic material and other macromolecules. For example, the dry solid medium of the invention can include a solid matrix and a composition which when applied to the dry solid medium protects against degradation of genetic material stored on the dry solid medium. The dry solid medium further provides for inactivation of microorganisms, including those which may be pathogenic to humans.

According to the invention, macromolecules stored on the dry solid medium may be analyzed using methods known in the art, for example, polymerized chain reaction (PCR), ligase chain reaction (LCR), reverse transcriptase initiated PCR, DNA or RNA hybridization techniques including restriction fragment length polymorphism (RFLP) and other techniques using genetic or DNA or RNA probes, genomic sequencing, enzymatic assays, affinity labeling, methods of detection using labels or antibodies and other similar methods.

In other embodiments, the invention provides methods for using the visible marker and dry solid media in the collection and tracking of biological materials in forms suitable for storage and subsequent analysis.

### BRIEF DESCRIPTION OF THE DRAWINGS

FIG. 1, illustration of a dry solid medium incorporating a diffusible dye in a grid pattern.

FIG. 2, illustration of the dry solid medium of FIG. 1 after the application of a sample and showing diffused dye in the portions of the grid pattern having the sample.

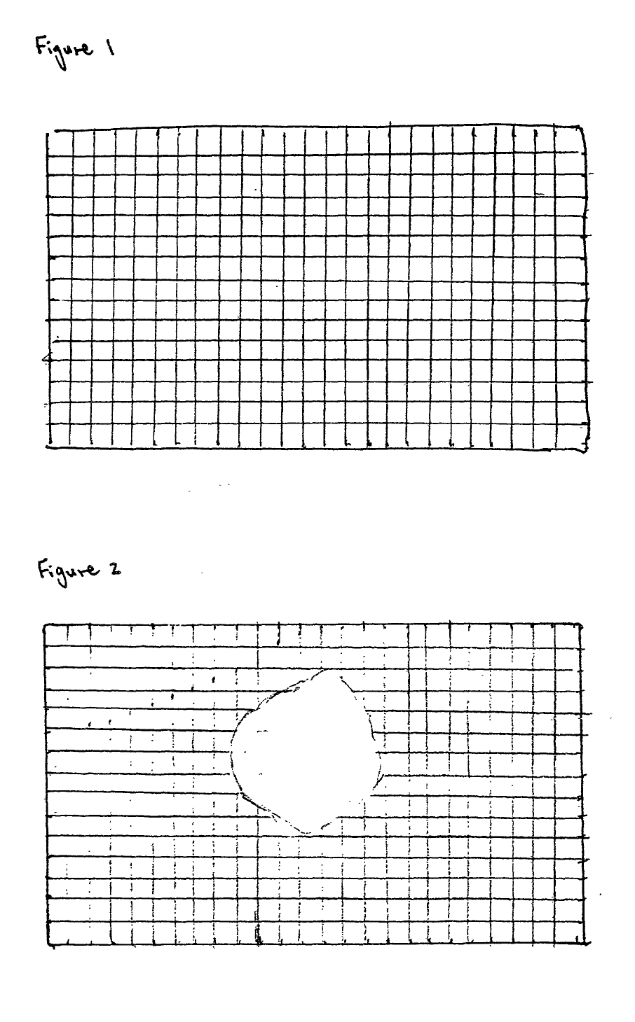

### DETAILED DESCRIPTION OF THE INVENTION

The present invention provides a dry solid medium with a visible tracer useful in the collection and tracking of biological materials in a form suitable for storage and subsequent analysis. The dry solid medium with a visible tracer and methods of use disclosed herein, provide a safe, convenient and reliable means for tracking and storing samples of genetic material and other macromolecules in a manner which provides reliable accuracy of analytical results. Moreover, the invention provides for enhanced convenience and efficiency when multiple samples are analyzed using automated systems, for example, at a centralized analyzing facility.

In several places throughout the present specification guidance is provided through example embodiments. The inventors wish to make clear that in each instance, the recited embodiments serve only as representative groups. It is not meant, however, that the embodiments are exclusive.

As used herein a “Biological sample” is used in its broadest sense and includes liquid or nonliquid samples from a wide variety of sources. Representative types of biological samples include tissue scrapings, whole blood, urine, cervical secretions, bronchial aspirates (including bronchial washings), sputum, saliva, feces, serum, synovial and cerebrospinal fluid, as well as laboratory preparations such as purified or partially purified macromolecules and cell culture materials.

As used herein, the term “visible” for example when used in a context such as “visible material” means a material which is discernible on the medium at some point during the practice of the invention, i.e. either before, after, or both before and after the application of a biological sample to the medium.

As used herein, “inert” is defined as molecules which have no deleterious interaction with macromolecules of interest within a sample and will not interfere with any subsequent analysis of the macromolecules.

As used herein, “nonspecific” is defined as molecules which do not target a specific macromolecule such a particular polypeptide or polynucleotide sequence, and which will not interfere with any subsequent analysis of these macromolecules.

As used herein “pattern” is used in its broadest sense and as used in the context of the visible material means a random or intentional design or configuration.

As used herein, the phrase “genetic material” (GM) means either or both deoxyribonucleic acid (DNA) or ribonucleic acid (RNA), or proteins in the case of proteinaceous infectious agents such as prions. According to the invention, a sample of GM is collected on the dry solid medium by removing the sample from a source and applying the sample to the herein described dry solid medium. Methods for removing a sample of genetic material from a source are known in the art. For example, a sample of genetic material in saliva may be removed from a human or animal source with a swab and the sample then applied to the dry solid medium of the invention.

As used herein a “sample of genetic material” or “sample of GM” includes a liquid having dissolved, suspended, mixed or otherwise contained therein, either or both DNA or RNA, cells which contain either or both DNA or RNA or cell components which contain either or both DNA or RNA. Once the sample of GM is applied to the dry solid medium, the liquid tends to evaporate (evaporation may be enhanced by a warm air dryer) leaving the DNA and/or RNA entrained to the dry solid medium in a dry form. The GM entrained to the dry solid medium in “dry form” may be purified DNA and/or RNA, semipurified DNA and/or RNA or DNA and/or RNA remaining in cells.

The sample containing macromolecules such as GM which is applied to the dry solid medium may be derived from any source. This includes, for example, physiological/pathological body liquids (e.g., secretions, excretions, exudates and transudates) or cell suspensions (e.g., blood, lymph, synovial fluid, semen, saliva containing buccal cells, skin scrapings, hair root cells, etc.) of humans and animals; physiological/pathological liquids or cell suspensions of plants; liquid products, extracts or suspensions of bacteria, fungi, plasmids, viruses etc.; liquid products, extracts or suspensions of parasites including helminths, protozoas, spirochetes, etc.; liquid extracts or homogenates of human or animal body tissues (e.g., bone, liver, kidney, etc.); media from DNA or RNA synthesis; mixtures of chemically or biochemically synthesized DNA or RNA; and any other source in which DNA and/or RNA is or can be in a liquid medium. Preferably, the liquid containing the GM evaporates after applying the sample to the dry solid medium leaving macromolecules including GM in dry form prior to subsequent analysis.

The dry solid medium of the invention provides for storage and/or subsequent analysis of the stored sample of GM or other macromolecules. The dry solid medium is composed of a solid matrix with a visible material that may be applied before or after a sample such that the visible material defines an area of the medium occupied by the sample. In addition to the visible material, the matrix of the dry solid medium can have sorbed thereto additional compositions such as a composition which can protect against degradation of the GM stored on the solid medium. Additional compositions can also enhance the purification of nucleic acids or may be employed to cause inactivation of microorganisms which may be associated with a sample of macromolecules including GM and which may be potentially pathogenic to human handlers of the stored sample.

A solid medium and a composition sorbed to a solid matrix is disclosed in U.S. Pat. No. 5,496,562, which has been incorporated herein by reference. As used herein, the term “storing”, “storage”, “stored” and other derivatives of “store”, when referring to macromolecules including GM in dry form entrained to the dry solid medium, means the preservation of GM in a form suitable for subsequent analysis and which has not undergone substantial degradation. The time period for which macromolecules including GM may be stored according to the invention may be as short as the time necessary to transport a sample from the place of collection of the sample to the place where subsequent analysis is to be performed. The conditions under which the sample of macromolecules including GM may be stored on the dry solid medium of the invention varies. Typically, samples are stored at temperatures from −200° C. to 40° C. In addition, stored samples may optionally be stored in dry or desiccated conditions or under an inert atmosphere. Storage may be for a few seconds up to many years, preferably, about 4 seconds as for robotic processing up to 100 years for database storage.

In another embodiment of the invention, the dry solid medium may further include a component which is functional in the subsequent analysis to be performed on the stored macromolecules. Subsequent analysis which may be performed on a sample stored on the dry solid medium includes analysis methods known in the art, for example, gel electrophoresis, polymerase chain reaction (PCR), ligase chain reaction (LCR), reverse transcriptase initiated PCR, DNA or RNA hybridization techniques including restriction fragment length polymorphism (RFLP) and other techniques using genetic or DNA or RNA probes, genomic sequencing, enzymatic assays, affinity labeling, methods of detection using labels or antibodies and other similar methods. In a preferred embodiment, the dry solid medium of the invention is a suitable medium for storage of components for subsequent analysis which are included on the dry solid medium. In addition, the inventors recognize that many new analytical and diagnostic methods may be developed in the future for which the dry solid medium and method of the invention may be equally useful and which would fall within the spirit and scope of the claims appended hereto.

In the case of stored RNA, particularly unstable RNA, components for subsequent analysis which may be included may also provide protection against RNA degradation. This includes RNase inhibitors and inactivators, proteins and organic moieties that stabilize RNA or prevent its degradation.

Once the macromolecules have been collected on the dry solid medium, the medium may be impregnated or encased in a protective material, for example, a plastic film, which may further protect against degradation during storage. Subsequent analysis of the macromolecules stored on the solid medium of the invention may be performed in situ on the solid medium or, alternatively, the macromolecules including GM may first be removed from the solid medium prior to subsequent analysis.

I. The Dry Solid Medium

The dry solid medium of the invention includes a visible diffusible marker sorbed to a solid matrix. As used herein, the term “sorb” means that the composition of the invention is absorbed, or otherwise incorporated into or onto the solid matrix in such a way as not to be readily removed from the matrix unless subjected to conditions which are intentionally or inadvertently performed to remove the sorbed composition from the solid matrix.

A solid matrix suitable for the dry solid medium and method of the invention includes any material to which the composition will sorb and which does not inhibit storage or subsequent analysis of the GM applied to the dry solid medium. This includes flat dry matrices or a matrix combined with a binder to form a pellet or tablet to which the composition is sorbed. In one preferred embodiment, the solid matrix is of a porous nature to provide entrainment of the macromolecules onto the dry solid medium. As used herein the term “entrain”, means that during storage the macromolecules are bound to the dry solid medium without substantial reliance on ionic, covalent or van der Waals interactions. A solid matrix suitable for this purpose includes, but is not limited to, a matrix which is cellulose based (e.g., cellulose, nitrocellulose or carboxymethylcellulose papers), hydrophilic polymers including synthetic hydrophilic polymers (e.g., polyester, polyamide, carbohydrate polymers), polytetrafluroethylene (EmporeJ, 3M, St. Paul, Minn.), fiberglass and porous ceramics.

Macromolecules may also be collected on a solid matrix which lacks the below-described composition of the invention. In addition, a component for subsequent analysis of a sample may also be included on a solid matrix which lacks the composition of the invention. Furthermore, hemoglobin or proteins associated with a sample of GM may be removed from a sample of GM stored on a solid matrix which does or does not include a component for subsequent analysis of the stored GM using an aqueous or nonaqueous (e.g., below-described single phase phenol wash) extraction procedure. However, by using only a solid matrix for storage, the GM protecting and pathogen inactivation effects of the composition of the invention obviously will not be available to the GM or components for subsequent analysis.

To form the dry solid medium of the invention, a composition which protects against degradation of macromolecules including GM is sorbed to the solid matrix. As used herein, the phrase “protects against degradation of GM” means that the dry solid medium of the invention maintains the stored GM in a substantially nondegraded form. This provides a sample of GM suitable for many different types of subsequent analytical procedures. Protection against degradation of GM may include protection against substantial damaging of GM due to GM damaging events such as that caused by chemical or biological means including action of bacteria, viruses, free radicals, nucleases, ultraviolet radiation, oxidizing agents and acidic agents (e.g., pollutants in the atmosphere).

Additional compositions sorbed to the solid matrix to form the dry solid medium of the invention may include one or more of a weak base, a chelating agent, a protein denaturant such as an anionic detergent, or a surfactant. In addition, the composition sorbed to the dry solid medium may also include a variety of additional molecules including free radical traps such as uric acid or a urate salt.

As used herein, the “weak base” of the composition may be a Lewis base which has a pH of about 6 to 10, preferably about pH 8 to 9.5. One function of the weak base is to act as a buffer to maintain a composition pH of about 6 to 10, preferably about pH 8.0 to 9.5, for example, pH 8.6. Hence, a weak base suitable for the composition of the invention may, in conjunction with other components of the composition, provide a composition pH of 6 to 10, preferably, about pH 8.0 to 9.5. Suitable weak bases according to the invention include organic and inorganic bases. Suitable inorganic weak bases include, for example, an alkali metal carbonate, bicarbonate, phosphate or borate (e.g., sodium, lithium, or potassium carbonate). Suitable organic weak bases include, for example, tris-hydroxymethyl amino methane (Tris), ethanolamine, triethanolamine and glycine and alkaline salts of organic acids (e.g., trisodium citrate). A preferred organic weak base is a weak monovalent organic base, for example, Tris. The Tris may be either a free base or a salt, for example, a carbonate salt.

Although the inventors do not wish to be limited to a single theory, it is believed that the weak base may provide a variety of functions, including protecting the GM from degradation. In addition to providing a buffer system, it is also believed that the weak base can act to ensure proper action of the below described chelating agent in binding divalent metal ions. In addition, the weak base may also prevent the action of acid nucleases which may not be completely dependent on divalent metal ions for functioning.

The composition of the dry solid medium can also include a chelating agent. According to the invention, a preferred chelating agent is a strong chelating agent. By “strong” chelating agent it is meant that the agent binds multivalent metal ions with a comparable or better affinity than ethylene diamine tetraacetic acid (EDTA). A preferred chelating agent according to the invention is EDTA. Although the inventors do not wish to be limited to a particular theory, it is believed that one function of the chelating agent of the invention is to bind divalent ions which if present with the stored GM may partake in causing damage to the GM. Ions which may be chelated by the chelating agent include divalent active metal ions, for example, magnesium and calcium, and transition metal ions, for example, iron. Both calcium and magnesium are known to promote GM degradation by acting as co-factors for enzymes which may destroy GM (e.g., most known nucleases). In addition, transition metal ions, such as iron, may readily undergo oxidation and reduction and damage nucleic acids by the production of free radicals or by direct oxidation.

The composition of the dry solid medium can further include an anionic detergent or surfactant. As used herein, the terms “surfactant” and “detergent” are synonymous and may be used interchangeably. Although not wishing to be limited to a single theory, it is believed that the anionic surfactant of the invention functions to denature non-GM compounds, for example, proteins, which are associated with the stored GM. Accordingly, denaturation of protein is one function of the anionic surfactant. According to the invention, any anionic surfactant which binds to and denatures proteins may be suitable for the invention. A preferred anionic detergent is a strong anionic detergent. As used herein, a “strong” anionic detergent includes a hydrocarbon moiety, aliphatic or aromatic, containing one or more anionic groups. Particularly preferred anionic detergents suitable for the invention include sodium dodecyl sulphate (SDS) and sodium lauryl sarcosinate (SLS). In a preferred embodiment, the anionic detergent of the invention causes inactivation of most microorganisms which have protein or lipids in their outer membranes or capsids, for example, fungi, bacteria or viruses. This includes microorganisms which may be pathogenic to humans and are present in a sample of GM.

Inactivation of a microorganism is believed to result from destruction of the secondary structure of its external proteins, internal proteins and any protein containing membranes necessary for viability. The inventors recognize that the anionic detergent may not inactivate some forms of organisms, for example, highly resistant bacterial spores and extremely stable enteric virions. Moreover, the inventors further recognize that while it may be desirable to inactivate pathogenic microorganisms, the GM of a microorganism associated with the stored sample of GM is also amenable for storage on dry solid medium of the invention. This allows for storage and/or subsequent analysis of the GM of a microorganism associated with a stored sample of GM.

The composition of the invention may optionally include a uric acid or a urate salt. According to the invention, the longer the period of time for which the GM is to be stored the more likely that uric acid or a urate salt may need to be included in the composition sorbed to the solid matrix. However, even if the GM is only to be stored for a matter of minutes, it may still be desirable to incorporate uric acid or urate salts into the composition.

While the inventors do not wish to be limited to any single theory, it is believed that the uric acid or urate salt may provide many functions. For example, the uric acid or urate salt may be converted to allantoin in acting as a free radical trap that preferentially accepts free radicals that would otherwise damage the nucleotide guanine. The free radicals are believed to be generated by spontaneous oxidation of the groups which are present, for example, in denatured serum protein of blood. Free radicals may also be generated due to oxidation or reduction of iron in blood. Because uric acid is a weak acid, it may also function as a component of the buffering system provided by the weak base as discussed above. In addition, the uric acid and urate salt may act as an erodible surface in that it is sparingly soluble so that a DNA sample dried onto its crystals will be released as the urate beneath erodes. Hence, the uric acid or urate salts may also provide for easy removal of a stored sample of GM if in situ processing is not desired.

Furthermore, after the sample of GM is applied to the dry solid medium, the dry solid medium with the applied sample of GM may be encased in a protective material, for example, a plastic film, which may further protect against degradation of stored GM. Examples of plastic films which are suitable according to the invention include polystyrene, polyethylene, polypropylene and other suitable lamination plastics. Encasing the dry solid medium in a protective material may be accomplished by methods known in the art. One simple method for encasing the dry solid medium in a plastic film is to put the dry solid medium into a container, e.g., a polyethylene bag, which is of sufficient size to hold the dry solid medium such that when a plastic film in liquid form is added to the container all parts of the dry solid medium will be coated by the liquid. The plastic film, in liquid form, is added to container to coat the dry solid medium. The liquid plastic film is allowed to dry to provide a plastic film coating which encases the dry solid medium. Prior to analysis, the plastic film is removed from the dry solid medium using methods known in the art, for example, dissolving with organic solvents such as chloroform or mechanical stripping.

The inventors further note that a dry solid medium including components for subsequent analysis, with an applied sample of GM, may also be encased in a protective material as described above.

II. Material for Sample Visualization on the Dry Solid Medium

The invention provides a dry solid medium for storing at least one biological sample, said dry solid medium comprising a pattern formed by a visible material, wherein upon application of a biological sample, the visible pattern is altered so that an area of the dry solid medium occupied by the sample is visible. For example, the dry solid medium of the invention can include a visible diffusible marker sorbed to a solid matrix. As used herein, the term “sorb” means that the composition of the invention is absorbed, adsorbed or otherwise incorporated into or onto the solid matrix in such a way as not to be readily removed from the matrix unless subjected to conditions which are intentionally or inadvertently performed to remove the sorbed composition from the solid matrix. Methods of absorbing, adsorbing or otherwise incorporating patterns of dyes and related materials into or onto a solid matrix are well known in the art, see e.g. U.S. Pat. Nos. 4,170,883 and 3,894,413. In the present invention, printing a pattern of dye or related material on the surface of warm, dry reagent-loaded paper by a press or by spraying through a template are the preferred method of getting such materials onto the solid medium.

The visible materials of the present invention address the problems associated with the fact that biological samples containing macromolecules for analysis are almost invisible on biological collection papers and related matrices. To overcome this inconvenience, the present invention discloses dry solid matrices manufactured with an agent that will indicate the area of the collection paper that has been wetted in the loading process. In this way, on application of a biological sample to surface of the matrix, the fluid in the sample will solubilize the visible material which will then diffuse with the liquid sample so that an area of the dry solid medium occupied by the liquid sample is visible against the background of nondiffused material. It will be apparent to one skilled in the art that the visible material may also be applied after the application of the sample to visualize the area of the medium covered by the sample.

In a typical embodiment of the invention, the visible agent is a dye such as bromophenol blue that has been applied to the dry solid medium in a specific pattern. On application of a biological sample to surface of the matrix, the fluid in the sample will dissolve the dye in the applied pattern and the dye will then diffuse so that the original printed pattern will disperse into the liquid and throughout the wet area until it dries. This dispersal will be confined to the wet areas and thus will make it clearly apparent where the matrix was wetted. When the pattern is printed only on the reverse side of the paper it can diffuse through the paper, from the reverse to the obverse side, resulting in a dyed area visible on the obverse surface.

The invention described herein has a variety of embodiments. One embodiment consists of a dry solid medium for storing at least one biological sample that includes a pattern formed by an inert visible material that, upon application of a biological sample, the is altered so that an area of the dry solid medium occupied by the sample is visible. In one version of this embodiment, the pattern formed by the inert visible material comprises a grid. In a preferred version of this embodiment, the visible material consists of inert, water-soluble dye molecules. In a preferred use of this embodiment, the biological sample consists of an aqueous sample. In a more preferred variation of this embodiment, the visible pattern is altered when the visible material diffuses in an aqueous biological sample.

Another embodiment consists of a dry solid medium for storing at least one biological sample, this dry solid medium containing an inert diffusible visible material which, upon application of a liquid sample, will diffuse with the liquid so that an area of the dry solid medium occupied by the liquid is visible. In a more specific embodiment of the invention, the dry solid medium consists of a cellulose based paper. In a preferred embodiment of the invention, the inert visible material consists of a pigment or dye such as bromophenol blue or carminic acid. In a highly preferred embodiment, the dye forms a pattern on the dry storage medium such as a grid, a checkerboard or a series of repetitive dots.

Another embodiment consists of a dry solid medium for storing at least one biological sample, this dry solid medium containing an inert diffusible visible material which, upon application of a liquid sample, will diffuse with the liquid so that an area of the dry solid medium occupied by the liquid is visible. In a more specific embodiment of the invention, the dry solid medium consists of a cellulose based paper. In preferred embodiment of the invention, the inert diffusible visible material consists of a dye such as bromophenol blue or carminic acid. In a highly preferred embodiment, the dye forms a pattern on the dry storage medium such as a grid, a checkerboard or a series of repetitive dots.

In another embodiment, the invention consists of a dry solid medium having a visible pattern formed by a diffusible nonspecific material, i.e. one that does not target a specific subset of macromolecules such a particular polypeptide or polynucleotide sequence. Upon application of a liquid sample, the nonspecific material diffuses with the liquid sample and alters the visible pattern so that an area of the dry solid medium occupied by the liquid sample is visible. In a more specific embodiment, the nonspecific material dye is selected from the group consisting of bromophenol blue, carminic acid, amino acridine or ethidium bromide. In a more specific embodiment, the dye forms a pattern such as a grid, a checkerboard or dots.

Additional embodiments include those where the solid medium includes both the visualization system disclosed herein and additional compositions which can interact with the macromolecules store on the solid medium. In one such embodiment, the invention consists of a solid medium for storage of at least one sample of DNA, a composition comprising a nonspecific visible material, wherein upon application of a biological sample, a property of the visible material is altered so that an area of the solid medium occupied by the biological sample is visible, an effective amount of a composition which protects against degradation of DNA adsorbed or incorporated onto the solid matrix, and additional compositions such as a protein denaturing agent and/or a free radical trap.

Yet another embodiment includes solid mediums having one of the variety of indicator systems as are well known in the art (see e.g. U.S. Pat. Nos. 5,491,094, 5,705,393, 5,185,247), the visualization system disclosed herein and additional compositions which can interact with the macromolecules store on the solid medium. In one such embodiment, the invention consists of a solid medium for storage of at least one sample of DNA, a chromogenic pH indicator, a visualization system, an effective amount of a composition which protects against degradation of DNA adsorbed or incorporated onto the solid matrix, and additional compositions such as a protein denaturing agent and/or a free radical trap.

Another embodiment of the invention consists of a method of visualizing a liquid biological sample by applying the sample to a dry solid medium having a visible pattern printed on it and formed by an inert, water-soluble dye, allowing the dye to diffuse with the liquid sample and alter the visible pattern; and visualizing the diffusion pattern of the dye that corresponds to an area of the dry solid medium occupied by the liquid sample.

There are a number of ways the visible agent may be applied to the dry solid medium including a variety of patterns. For example, the visible material may be applied to a paper like matrix in grid patterns with lines spaced at a specific distance such as 3 mm. Alternatively, the visible material may be applied in patterns of dyed squares, with alternating un-dyed squares. Patterns of visible material may also be an array of extremely fine but intensely stained dots set at some specific distance, for example 2 mm, apart. Alternatively, patterns may consist of a repetitive word such as a company name, or repetitive design such as a company logo. In addition, patterns may be applied on either the obverse or reverse side of a paper like matrix. Moreover, those skilled in the art appreciate that there are a potentially infinite variety of patterns that may be applied, and the patterns illustrated above comprise a small subset of representative designs.

While the use of diffusion is preferred, those skilled in the art appreciate that there a wide variety of ways to induce chromogenic changes in a substrate when this substrate comes into contact with a biological sample. Therefore, the practice of the invention does not necessarily include diffusible visible materials. For example with pH indicator papers, it is routine to induce pH mediated chromogenic changes using covalently bound pH sensitive indicator dyes applied to a layer of cellulose particles on one side of an indicator paper. Such papers are prepared by placing a thin layer of cellulose on one side of the paper, impregnating the paper with alkaline reagents and then spraying the a thin layer of the surface of the paper with an acid in a non-aqueous solvent such as ethanol, acetone or a appropriate hydrocarbon so that the thin layer with the bound dye is acidic while the rest of the paper is strongly alkaline. Upon wetting, the basic constituents move into the thin acid layer, resulting in the indicators dye's changing from one color to another. A wide variety of these types of pH based indicator systems are well known in the art, see e.g. U.S. Pat. Nos. 4,029,598, 4,824,827 and 4,699,885.

Those skilled in the art will appreciate that there are a wide variety of substances that provide materials useful in the claimed invention. As illustrated above, non-fast, water-soluble dyes that will not interfere with subsequent biological analyses are included in the preferred embodiments of the invention. Bromophenol blue ion and carminic acid ion (cochineal) dyes (or molecules such as their sodium or tris salts) are illustrative dyes that may be used for this purpose. Additional dyes include ethidium bromide and aminoacridine, nucleic acid dyes which are well known in the art, see e.g. U.S. Pat. No. 5,599,932. Bromophenol blue is well known in the art and an example of its use in a solid matrix is disclosed in U.S. Pat. No. 5,049,358 to Lau et al. In addition, carminic acid (cochineal) dyes are well known in the art as disclosed in U.S. Pat. No. 5,147,673 to Schul. These dyes are favored because they are non-toxic and non-interfering with most molecular chemistry. Those skilled in the art appreciate that there are a wide variety of dyes that are useful in this invention and that the examples of Bromophenol blue and carminic acid are provided as illustrative embodiments and are not intended to limit the invention in any way.

In addition to the dyes described above, additional substances may provide diffusible materials useful in the claimed invention. For example, metal or carbon sol particles may be useful in accordance with the present invention. Preferably, the detectable species may be a metal-containing particle of the sort fully described in U.S. Pat. No. 4,859,612. In accordance with the concepts and principles of the present invention, metal sol particles having a particle size in the range of from about 50 to about 1000 Angstroms. Such metal particles, and in particular gold sol coated with proteins on their surface have already been described by M. Horisberger et al. in Experimentia, 31, pp. 1147-1149, Oct. 15, 1975. Such particles are intensely colored, either orange, red or violet, depending on particle size. The metal sol particles to be used in accordance with the present invention may be prepared by methodology which is known. For instance, the preparation of gold sol particles is disclosed in an article by G. Frens, Nature, 241, 20-22 (1973). Additionally, the metal sol particles may be metal or metal compounds or polymer nuclei coated with metals or metal compounds, all as described in U.S. Pat. No. 4,313,734 to Leuvering. In this regard, the metal sol particles may be of platinum, gold, silver or copper or any number of metal compounds which exhibit characteristic colors.

Other substances may also provide diffusible materials useful in the claimed invention. Dyed latex polymers, such as blue, red, green, orange, or yellow latex polymer particles, may be incorporated into the dry solid matrix of the present invention. It is well known in the art that latex polymer particles can be dyed. While it is well known in the art that protein substances (including carrier proteins such as bovine serum albumin) can be coupled to latex polymer particles, in the favored embodiments of the present invention it is preferred that the latex particle are not coupled to any macromolecules. Latex molecules well known in the art include styrene-glycidyl methacrylate (SGM) latex colored with a dye. Materials substantially equivalent to styrene-glycidyl methacrylate listed in U.S. Pat. No. 4,210,723 are acceptable latex polymer particles that can also be used in the practice of this invention.

In addition to the specific embodiments discussed above, there are a number of variations of disclosed material embodiments that may be used in the present invention. For example, the various latex and/or plastic granules could contain a pH sensitive dye in its acidic state and this plastic bound dye can then be imprinted onto the surface of a dry, reagent loaded paper by a heat printing process. On wetting, the reagents would then come into contact with the plastic, and produce a color change. In particular, by using plastic materials that are slightly water soluble or contain some plastic soluble agent that can communicate the external pH of a fluid (the pH of the paper) into the interior of the plastic, the presence of a liquid such as water would then cause the plastic granules to change color as the external pH is communicated into the center of the plastic granules.

The present invention is further detailed in the following Examples, which are offered by way of illustration and are not intended to limit the invention in any manner. All patent and literature references cited in the present specification are hereby incorporated by reference in their entirety.

### EXAMPLES

Example 1

This example illustrates a preferred embodiment of a sample collection medium incorporating a visible diffusible material for sample visualization.

A cellulose paper having sorbed thereto a carminic acid ion dye printed in a grid pattern on its reverse side. The grid pattern forms a series of repetitive squares produced by perpendicular lines spaced 3 millimeters apart. On application of a biological sample to the paper surface, the liquid in the sample dissolves the dye in the grid pattern and diffuses so that the dye in the wetted portions of the grid pattern is dispersed in the liquid and throughout the wet area until it dries. As this dispersal is confined to the wetted areas, this dispersal of the dye makes the boundaries of the diffused liquid sample clearly apparent.

As the dye dispersal pattern corresponds to the applied biological sample, this dispersal pattern allows the technician to focus only those portions of the biological collection paper having molecules of interest. In this way, this visible representation of the liquid sample facilitates the processing and analysis of the biological specimen.

As many modifications and variations can be made in the above methods and products without departing from the spirit and scope of the invention, it is intended that all matter contained in the above description or shown in the accompanying drawing shall be interpreted as illustrative and not in a limiting sense.

What is claimed is:

1. A dry solid medium for storing at least one biological sample, said dry solid medium comprising a material that enables the visualization of the sample applied to the medium, wherein said material does not interfere with analysis of the sample.

2. The dry storage medium of claim 1, wherein the material comprises a pigment or dye.

3. The dry storage medium of claim 1, wherein the pigment or dye is selected from the group consisting of carbon or metal sols, bromophenol blue and carminic acid.

4. The dry storage medium of claim 2, wherein the pigment or dye forms a pattern on the dry storage medium.

5. The dry storage medium of claim 4, wherein the pattern formed by the pigment or dye comprises a grid.

6. The dry storage medium of claim 4, wherein the pattern formed by the pigment or dye comprises a checkerboard.

7. The dry storage medium of claim 4, wherein the pattern formed by the pigment or dye comprises dots.

8. A dry solid medium for storing at least one biological sample, said dry solid medium comprising a visible material having an appearance wherein upon application of a biological sample, the appearance of the visible material is altered so that an area of the dry solid medium occupied by the biological sample is visible.

9. The dry storage medium of claim 8, wherein the visible material is in a pattern which is altered by diffusion.

10. The dry storage medium of claim 9, wherein the visible material comprises an inert, water-soluble dye.

11. The dry storage medium of claim 10, wherein the dye is selected from the group consisting of bromophenol blue and carminic acid.

12. The dry storage medium of claim 8, wherein the visible material is in a pattern formed by the dye and the pattern comprises a grid.

13. The dry storage medium of claim 10, wherein the pattern formed by the dye comprises a checkerboard.

14. The dry storage medium of claim 10, wherein the pattern formed by the dye comprises dots.

15. A dry solid medium for storage of a sample of genetic material which is to be subsequently analyzed, said dry solid medium having sorbed thereto a composition comprising a nonspecific visible material, wherein upon application of a biological sample, a property of the visible material is altered so that an area of the dry solid medium occupied by the biological sample is visible.

16. A solid medium for storage of at least one sample of DNA, the solid medium comprising:
(a) a solid matrix;
(b) a composition comprising a nonspecific visible material, wherein upon application of a biological sample, a property of the visible material is altered so that an area of the solid medium occupied by the biological sample is visible; and
(c) an effective amount of a composition which protects against degradation of DNA adsorbed or incorporated onto said solid matrix, said composition comprising:
(i) a protein denaturing agent; and
(ii) a free radical trap.

17. A method of visualizing a liquid biological sample comprising the steps of:
(a) applying the biological sample to a dry solid medium used for storing at least one biological sample, said dry solid medium comprising a visible material formed by including an inert, water-soluble dye;
(b) allowing the dye to diffuse with the liquid sample and alter an appearance of the visible material; and
(c) visualizing the diffusion appearance of the dye that corresponds to an area of the dry solid medium occupied by the liquid sample.

18. A method of visualizing a liquid biological sample comprising the steps of:
(a) applying the sample to a dry solid medium used for storing at least one biological sample;
(b) allowing the sample to diffuse through the solid medium;
(c) applying an inert water-soluble dye to the solid medium to form an appearance; and
(d) visualizing the appearance of the dye that corresponds to an area of the dry solid medium occupied by the liquid sample.
